(19) United States
(12) Patent Application Publication
(10) Pub. No.: US 2006/0003980 A1
(43) Pub. Date: Jan. 5, 2006

(54) Cobalt(II) complexes as protein tyrosine kinase inhibitors

(76) Inventors: Shamsuddin Shaikh, Houston, TX (US); Raymond J.A. Budde, Bellaire, TX (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/884,067

(22) Filed: Jul. 2, 2004

(51) Int. Cl. C07F 15/06; A61K 31/555

(52) U.S. Cl. 514/184; 548/101; 548/402

(57) ABSTRACT

Various cobalt (II) complexes are able to selectively inhibit the protein tyrosine kinases (PTKs). These complexes are useful in the treatment of various diseases including hyperproliferative diseases, hematologic diseases, osteoporosis, neurological diseases, autoimmune diseases, allergic/immunological diseases, or viral infections.

### BACKGROUND

1. Technical Field

Novel complexes are disclosed which are useful for the treatment of diseases related to increased protein tyrosine kinase activity. Methods of synthesis of these complexes and methods of treatment employing these complexes are also disclosed. The novel complexes include cobalt (II) complexes capable of inhibiting the protein tyrosine kinases (PTKs).

2. Background of the Related Art

Within the Src family of PTKs, Src is associated with cellular membranes and is involved in signal transduction and growth regulation pathways (Sridhar and Cooper, 2000, Frame, 2002). Src propagates cellular signals by transferring the gamma phosphate of ATP to the side chain of tyrosine residues on substrate proteins. Alterations in the phosphorylation of Src substrates are key events in cellular signaling. Most normal cells contain very low levels and activity of Src (Barnekow, 1989) and the Src enzyme is not required for the establishment or maintenance of cell viability (Sariano, et al., 1991).

However, excessive Src activity is associated with various cancers, and therefore Src is a drug target in oncology (Cartwright et al., 1990). For example, Src activity is greatly increased in breast cancer (Partanen, 1994); stomach cancer (Takeshima et al., 1991); colon cancer (Termuhlen et al., 1993); hairy cell leukemia and a subgroup of B-cell lymphomas (Lynch et al., 1993); low grade human bladder carcinoma (Fanning et al., 1992); neuroblastoma (Bjelfman et al., 1990); ovarian cancer (Wiener et al., 1999); and non-small cell lung carcinoma (Budde et al., 1994). In the case of colon cancer, Src is activated more frequently than Ras or p53 (Jessup et al., 1993). Src undergoes two distinct activations corresponding with malignant transformation of colonocytes (Cartwright et al., 1990) and tumor progression (Termuhlen et al., 1993).

Antisense to Src inhibits growth of human monoblastoid leukemia cells (Waki et al., 1994), K562 human leukemia cells (Kitanaka et al., 1994) and HT-29 human colon cancer cells (Staley et al., 1997). Src activity has been reduced in a human ovarian cancer cell line (SKOv-3) by antisense technology. The reduced Src activity in SKOv-3 is associated with altered cellular morphology, reduced anchorage-independent growth, diminished tumor growth and reduced vascular endothelial growth factor mRNA expression in vitro (Wiener et al., 1999).

Inhibition of Src would have the effect of interrupting the signal transduction pathways in which it participates and would thereby reduce the rate of growth of cancer cells.

Src inhibitors are currently being studied for use in the treatment of hematologic and solid tumors, inflammatory and autoimmune diseases (Sinha et al., 1999). Src inhibitors have potential for treatment of osteoporosis, a condition in which bone resorption is increased resulting in weakening of bone. It was shown that mice depleted of the Src gene developed osteopetrosis (Sariano et al., 1991) and that Src is involved with bone resorption (20).

Potential sites for targeting inhibitors of Src family PTKs are the SH2 and SH3 domains (Park et al., 2003), the phosphoryl transfer site (SH1 domain), i.e., the active site or other unknown sites on the enzyme. Compounds binding to SH2 and SH3 domains would block the protein-protein interactions and the recruitment of other signal transduction proteins mediated by these domains. Active-site directed inhibitors could be targeted to the ATP binding site, the protein substrate binding site, or both (bisubstrate analogues).

The role of magnesium ions relative to catalysis by PTKs is being studied and it has been demonstrated that PTKs typically require two magnesium ions (Sun et al., 1997). One magnesium ion is used by all PTKs to complex with ATP and the second magnesium ion is essential for catalysis (Id.).

For some receptor-type PTKs, the second magnesium ion forms a bidentatae complex with ATP-Mg (i.e., ATP-[Mg]2) to reduce the Km for ATP to physiological values. However, with other PTKs, such as the Src and Csk families, the second magnesium ion binds freely to the apoenzyme and does not affect the Km for ATP-Mg.

While substitution studies have demonstrated that these magnesium ions can be replaced with higher affinity (10,000-fold) metals, the issues of stability, non-toxicity, physiological compatibility, affinity and selectivity have yet to be addressed.

### SUMMARY OF THE DISCLOSURE

In satisfaction of the aforenoted needs, disclosed herein are a number of small-molecule cobalt (II) PTK inhibitors that are suitable to act as pharmaceuticals. The inhibitors disclosed herein may be targeted to the phosphoryl transfer site (SH1 domain), i.e., the active site. Active-site directed inhibitors can be targeted to the ATP binding site, the protein substrate binding site, or both (bisubstrate analogues). While the disclosed complexes serve as inhibitors for the Src family of PTKs, it will be understood that the disclosed complexes may very well serve as inhibitors to additional families of PTKs or other protein kinases as well.

The disclosed complexes are selected from the following general formulas: 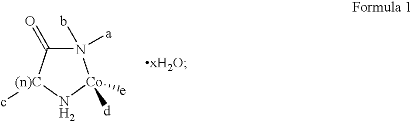
wherein a is H or —;
wherein b is H or OH;
wherein c is selected from the group consisting of H, —CH₃, 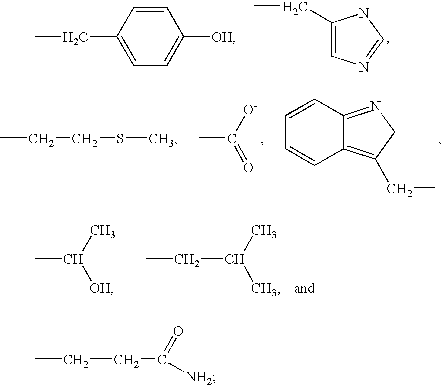
wherein d is selected from the group consisting of OH, ClO₄, imidazole, methionyl, carboxylic, tryptophenyl, threonyl, amide and carbonate;
wherein e is selected from the group consisting of OH, ClO₄, H₂O, Cl, 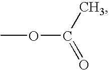
Cl₂, and carbonate; and
wherein n is an integer ranging from 1 to 3; and
wherein x ranges from 0 to about 3;
Formula 2 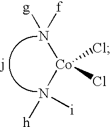
wherein f, g, and h are H or —;
i is H, —, or 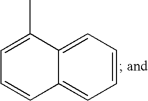
j is —CH₂CH₂—, 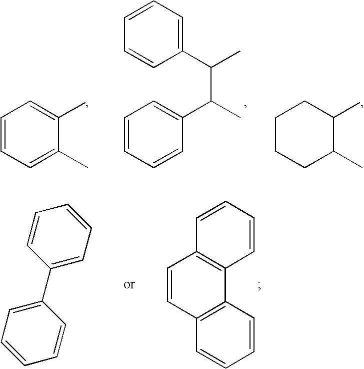 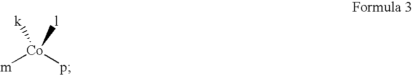
wherein k and l are H₂O or Cl; and
m and p are; 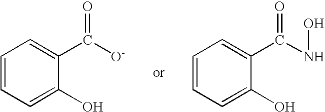 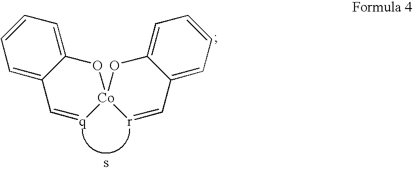
wherein q and r are N, O or 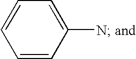
wherein s is —CH₂CH₂—, —, or

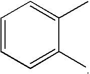

In a further embodiment, the PTK inhibiting cobalt complex may be selected from the following group of exemplary complexes:
Co(L-Tyrosinehydroxamate)(H₂O)₂(ClO₄)₂;
Co(L-Tyrosine amide)(H₂O)₄(ClO₄); Co(L-Histidinehydroxamate)(H₂O)(ClO₄);
Co(DL-Methioninehydroxamate)(OH)H₂O;
Co(Glycinehydroxamate)(OH)₂; Co(DL-Asperticacid-β-hydroxamate)(H₂O)₃;
Co(L-Asperticacid-β-hydroxamate)(H₂O)₃; Co(β-alaninehydroxamate)(OH)₂; Co(DL-alaniniehydroxamate)(OH)₂;
Co(L-tryptophanhydroxamate)(H₂O)(ClO₄); Co(D-tryptophanhydroxamate)(H₂O)(ClO₄);
Co(L-Threoninehydroxamate)(OH)(H₂O); Co(L-Tryptophanamide)(H₂O)₂(ClO₄)₂;
Co(L-Tyrosineamide)(H₂O)₃Cl₂;
[Co(L-Tyrosine amide)(OH)Cl].1½H₂O; Co(L-Tyrosine amide)(Acetate)(OH);
[Co(glycinamide)(H₂O)₂Cl₂].NaCl; Co(L-Leucinamide)Cl₂;
Co(D-leucinamide)(H₂O)₂Cl₂;[Co(L-glutamine)Cl].1/2 H₂O;
[Co(D-glutamine)Cl].H₂O;[Co(L-glutamine-α-amide)Cl₂].3H₂O;
Co(L-tyrosineamide)(CO₃)H₂O; Co[N,N-bsi(salicylidene)ethylenediamine];
Co[N,N-bis(salicylidene)1,2-phenylenediamine];
Co(triphenylphosphine)₂Cl₂;
Co[1,2-bis(diphenylphosphino)ethane]Cl₂;
Co[N,N-bis(salicylidene)dianiline];
Co[bis(salicylideniminaato-3-propyl)methylamine];
Co(acetylacetanote)₂;
Co(hexafluoroacetylacetonate)₂;
Co(benzoylacetonate)₂;
Co(salicylaldehyde)₂;
Co(ethylenediamine)(OH)Cl;
Co(1,2-phylenediamine)Cl₂;
[Co(N-naphthylethylenediamine)Cl₂].H₂O;
{Co[(Meso-1,2-diphenyl)ethylene-diamine]Cl₂}.½H₂O;
Co(2,2′-dipyridyl)Cl₂; Co(1,10-phenanthroline)Cl₂;
[Co(trans-1,2-DACH)Cl₂].½H₂O;
{Co[(R,R-1,2-diphenyl)ethylene-diamine]Cl₂}.½H₂O;
Co(Salicylate)₂(H₂O)₂; Co(Salicylhydroxamate)₂Cl₂;
Co(Thiosalicylate)₂(H₂O)₂; and mixtures thereof.

A further embodiment is a pharmaceutical composition for the treatment of human and mammal diseases including but not limited to hyperproliferative diseases, hematologic diseases such as osteoporosis, neurological diseases such as Alzheimer's Disease, epilepsy or senility, autoimmune diseases, allergic/immunological diseases such as anaphyiaxis, or viral infections which comprises a pharmaceutically acceptable carrier and a therapeutically effective amount of at least one cobalt complex disclosed herein or a pharmaceutically acceptable salt or hydrate thereof. The uses of the disclose PTK inhibiting cobalt complexes are not limited to the diseases listed herein.

Another embodiment is a method of synthesizing one or more of the cobalt complexes: disclosed. Synthesis procedures are explained in detail below.

Another embodiment is a method of inhibiting PTKs by administering to a subject one or more cobalt complexes disclosed herein.

In a further embodiment, the step of the binding at least one of the disclosed cobalt complexes to protein tyrosine kinases may be included. In a further embodiment, the cell may be contacted with one or more of the disclosed cobalt complexes in order to alter cell morphology, migration, adhesion, cell cycle progression, secretion, differentiation, proliferation, anchorage-independent growth, vascular endothelial growth factor expression, microtubule binding by tau, viral infectivity, or bone reabsorption.

Another embodiment is a method of treating a PTK-related disease in a subject comprising the step of administering to the subject a pharmaceutically acceptable carrier and a therapeutically effective amount of one or more of the disclosed cobalt complexes.

In further embodiments, the administering may parenteral. In still further embodiments, the parenteral administration may be intravenous, intramuscular, subcutaneous, intraperitoneal, intraarterial, intrathecal or transdermal. In a further embodiment, the administering may be alimentary. In a further embodiment, the alimentary administration may be oral, rectal, sublingual, or buccal. In a further embodiment, the administration may be topical. In a further embodiment, the administration may be by inhalation. In a further embodiment, the administering may be combined with a second method of treatment.

Another embodiment is a method of preventing replication of a virus in an organism by administering to the organism infected with the virus one or more of the cobalt complexes disclosed herein. In a further embodiment, the virus may be a herpesvirus, papovavirus, hepadnavirus or retrovirus.

As used herein the specification, “a” or “an” may mean one or more. As used herein in the claim(s), when used in conjunction with the word “comprising,” the words “a” or “an” may mean one or more than one. As used herein “another” may mean at least a second or more.

Other features and advantages of the disclosed cobalt complexes, synthesis methods and treatment methods will become apparent from the following detailed description. It should be understood, however, that the detailed description and the specific examples, while indicating certain preferred embodiments, are given by way of illustration only, since various changes and modifications that fall within the spirit and scope of this disclosure will become apparent to those skilled in the art from this summary and the following detailed description.

### BRIEF DESCRIPTION OF THE DRAWING

FIG. 1 graphically illustrates that a stable cobalt-tyrosineamide identified as complex no. 2 in Table 1 causes the cell growth inhibition in contrast to free metal or the tyrosine amide which are shown to be not inhibitory.

### DETAILED DESCRIPTION OF THE PRESENTLY PREFERRED EMBODIMENTS

PTKs catalyze the transfer of the gamma phosphate of ATP to protein substrates within the cell. The disclosed cobalt (II) complexes act as inhibitors to this process by blocking this transfer of the phosphate thereby inhibiting the catalytic activity of PTKs. By blocking the catalytic activity of PTKs, the signal-transduction pathway regulating growth can be stopped or significantly impeded.

Definitions

Hematologic Disease As used herein, “hematologic disease” refers to a disease in which there is abnormal generation of blood cells.

Neurologic Disease As used herein, “neurologic disease” refers to a disease caused by abnormalities within the nervous system.

Proliferative Disease As used herein, “proliferative disease” refers to a disease that occurs due to abnormal growth or extension by the multiplication of cells.

Autoimmune Disease As used herein, “autoimmune disease” refers to a disease caused by the presence and activation of T or B lymphocytes capable of recognizing “self” constituents with the release of auto-antibodies or damage caused to cells by cell-mediated immunity.

Allergic/Immunological Disease As used herein, “allergic/immunological disease” refers to disease caused by one or more aspects of the immune system. Examples of included types of diseases are immunodeficiency, characterized by increased susceptibility to infections due to the deficiency of a component of the immune system (B cells, T cells, phagocytic cells, and complement); hypersensitivity disorders, which result from immunologically specific interactions between antigens (exogenous or endogenous) and humoral antibodies or sensitized lymphocytes; and reactions to transplantations, in which allografts are rejected through either a cell-mediated or a humoral immune reaction of the recipient against antigens present on the membranes of the donor's cells.

Viral Infection As used herein, “viral infection” refers to a disease caused by the invasion of body tissue by a micro-organism that requires a cell in which to multiply.

Binding As used herein, “binding” refers to the non-covalent or covalent interaction of two chemical complexes.

Inhibiting As used herein, “inhibiting” refers to the ability of a substance to reduce the velocity of an enzyme-catalyzed reaction. A substance is a better inhibitor than another if it is able to cause the same amount of reduction in velocity at a lower concentration than another substance.

Orieniation of Complexes

Certain disclosed complexes or complexes may exist in different enantiomeric forms. This disclosure relates to the use of all optical isomers and stereoisomers of the disclosed complexes that possess the desired activity. One of skill in the art would be aware that if a given isomer does not possess the desired activity, that isomer should not be used for treatment.

Pharmaceutical Compositions

Pharmaceutically Acceptable Carriers

The disclosed complexes comprise an effective amount of one or more disclosed complexes dissolved and/or dispersed in a pharmaceutically acceptable carrier.

The phrases “pharmaceutically and/or pharmacologically acceptable” refer to molecular entities and/or compositions that do not produce an adverse, allergic and/or other unacceptable reaction when administered to an animal.

As used herein, “pharmaceutically acceptable carrier” includes any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For human administration, preparations should meet sterility, pyrogenicity, general safety and/or purity standards as required by FDA Office of Biologics standards. Various pharmaceutical preparations and administration methods are discussed in U.S. Pat. No. 6,503,914 and the references cited therein.

Lipid Formulations and/or Nanocapsules

In certain embodiments, the use of lipid formulations and/or nanocapsules is contemplated for the introduction of with the disclosed complexes into host cells as disclosed in U.S. Pat. No. 6,503,914.

Kits

Disclosed therapeutic kits comprise the disclosed complexes or pharmaceutically acceptable salts thereof. Such kits will generally contain, in suitable container means, a pharmaceutically acceptable formulation of with the disclosed complexes in a pharmaceutically acceptable formulation as disclosed in U.S. Pat. No. 6,503,914. The kit may have a single container means, and/or it may have distinct container means for each complex.

Combination Treatments

In order to increase the effectiveness of with the disclosed complexes, it may be desirable to combine these compositions with other agents effective in the treatment of the disease as disclosed in U.S. Pat. No. 6,503,914. The disclosed complexes may also be combined with other agents, treatments and/or therapies in the treatment of hematologic diseases, osteoporosis, neurological diseases, autoimmune diseases, allergic/immunological diseases, viral infections, and hyperproliferative disease. Such treatments and therapies that may be combined with the use of the disclosed complexes include chemotherapy, radiotherapy, immunotherapy, gene therapy, antisense, inducers of cellular proliferation, inhibitors or cellular proliferation, regulators of programmed cell death, surgery and other agents and treatment as discussed in U.S. Pat. No. 6,503,914, the references cited therein and the references cited herein.

The disclosed cobalt complexes theoretically have a high affinity to the second metal binding site (Id.) and should be relatively non-toxic and physiologically compatible (Curtis et al., 1976). The disclosed hydroxamate structures are also theoretically non-toxic and form very stable complexes with metals (Hall et al., 2002; Brown et al., 2001; and O'Brien et al., 2000). Hydroxamates are often used as drugs that inhibit metalloporteases (Drugs of the Future, 1996). Furthermore, the disclosed cobalt compleses are promising as cobalt is non-toxic and is routinely given therapeutically (Bowie et al., 1975; Valberg, 1971; and Edwards et al., 1971).

### EXAMPLES

Disclosed Cobalt complexes were prepared with metal to ligand ratio ranging from about 1:1 to about 1:2. This ratio was achieved by using absolute ethanol as a reaction medium. The ratio was confirmed by the elemental analyses of the complexes and as shown in Table 1, the theoretical values are in good agreement with the actual findings. Some Cobalt(II) complexes were purchased from Aldrich Chemical Company and all complexes were screened against Src, Csk and FGF-r as shown in Table 2.

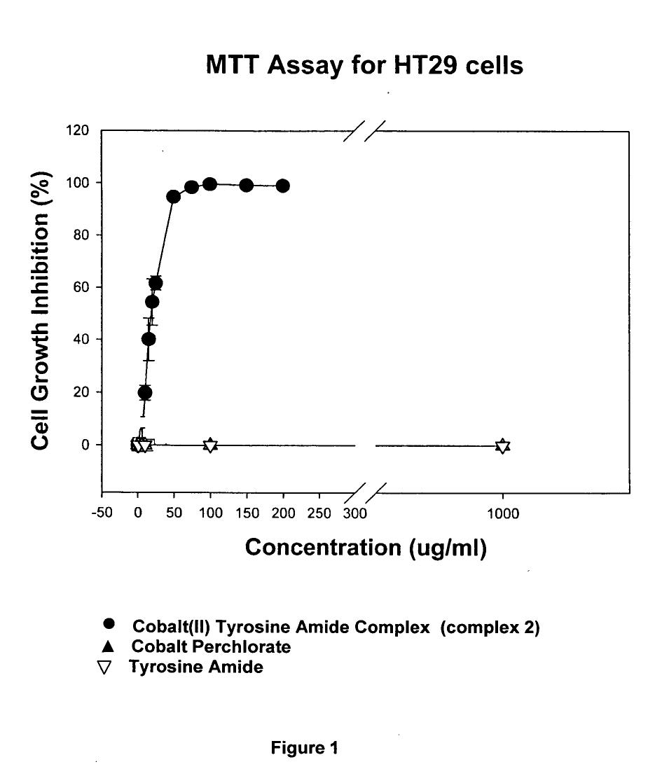

FIG. 1 shows proof of the concept that a stable cobalt-tyrosine amide complex (No. 2 in Table 1) causing the cell growth inhibition. In contrast, neither free metal nor the tyrosine amide were inhibitory. This indicates a requirement for both the tyrosine amide moiety and for the metal moiety.

General Synthetic Procedure of Cobalt(II) Complexes

1 mM of ligand was suspended in 20 ml of absolute ethanol and an equivalent amount of ethanol solution (1M) of CoCl_{2.}6H₂O was added drop wise to the reaction mixture while stirring at room temperature. The pH of the mixture was adjusted to 8 with 1 N NaOH solution in ethanol. Stirring was continued for 24 hours. A fine precipitate separated from the solution was isolated by centrifugation, washed thoroughly with ethanol and ether and dried over P₂O₅ under vacuum.

Complexes 1-13 were prepared with Co(ClO₄)₂.6H₂O.

Complex 16 was prepared with Co(acetate)₂.4H₂O

Complex 23 was prepared with CoCO₃.

Complexes 1, 10, 18, 19, and 23 were isolated by precipitation the concentrated reaction mixture with ether.

Complexes 14 and 16 were precipitated with acetone.

Complexes 35-44 were purchased from Aldrich Chemical Company and, hence, elemental analyses of these compounds were not separately performed.

TABLE 1
Elemental Analyses of the Synthesized Cobalt(II) Complexes

| | | Found (Calculated) | | |
|---|---|---|---|---|
| No. | Complex | % C | % H | % N |
| 1. | Co(L-Tyrosinehydroxamate)(H₂O)₂(ClO₄)₂ | 22.05 | 3.29 | 5.72 |
| | | (22.47) | (2.94) | (5.94) |
| 2. | Co(L-Tyrosine amide)(H₂O)₄(ClO₄) | 26.39 | 4.64 | 6.84 |
| | | (26.72) | (4.15) | (6.37) |
| 3. | Co(L-Histidinehydroxamate)(H₂O)(ClO₄) | 20.84 | 3.18 | 16.20 |
| | | (20.80) | (3.47) | (16.03) |
| 4. | Co(DL-Methioninehydroxamate)(OH)H₂O | 23.22 | 4.51 | 9.55 |
| | | (23.35) | (5.45) | (10.89) |
| 5. | Co(Glycinehydroxamate)(OH)₂ | 13.26 | 4.78 | 15.13 |
| | | (13.11) | (4.37) | (15.30) |
| 6. | Co(DL-Asperticacid-β-hydroxamate)(H₂O)₃ | 18.58 | 4.64 | 10.34 |
| | | (18.53) | (4.63) | (10.81) |
| 7. | Co(L-Asperticacid-β-hydroxamate)(H₂O)₃ | 18.40 | 4.50 | 10.33 |
| | | (18.53) | (4.63) | (10.81) |
| 8. | Co(β-alaninehydroxamate)(OH)₂ | 17.89 | 5.15 | 14.52 |
| | | (18.27) | (5.17) | (14.22) |
| 9. | Co(DL-alaniniehydroxamate)(OH)₂ | 18.71 | 4.99 | 14.16 |
| | | (18.27) | (5.17) | (14.22) |
| 10. | Co(L-tryptophanhydroxamate)(H₂O)(ClO₄) | 33.28 | 3.68 | 9.97 |
| | | (33.46) | (3.55) | (10.65) |
| 11. | Co(D-tryptophanhydroxamate)(H₂O)(ClO₄) | 33.62 | 4.29 | 10.55 |
| | | (33.46) | (3.55) | (10.65) |
| 12. | Co(L-Threoninehydroxamate)(OH)(H₂O) | 21.65 | 4.81 | 12.61 |
| | | (21.15) | (5.28) | (12.33) |
| 13. | Co(L-Tryptophanamide)(H₂O)₂(ClO₄)₂ | 26.62 | 3.50 | 8.17 |
| | | (26.62) | (3.24) | (8.46) |
| 14. | Co(L-Tyrosineamide)(H₂O)₃Cl₂ | 32.02 | 5.19 | 8.07 |
| | | (32.55) | (5.46) | (8.40) |
| 15. | [Co(L-Tyrosine | 35.96 | 4.58 | 8.91 |
| | amide)(OH)Cl].1½H2O | (35.72) | (5.20) | (9.26) |
| 16. | Co(L-Tyrosine amide)(Acetate)(OH) | 41.66 | 5.72 | 8.85 |
| | | (41.77) | (5.42) | (8.88) |
| 17. | [Co(glycinamide)(H₂O)₂Cl₂]; NaCl | 8.14 | 3.32 | 8.73 |
| | | (8.05) | (3.38) | (9.28) |
| 18. | Co(L-Leucinamide)Cl₂ | 27.69 | 6.09 | 10.13 |
| | | (27.70) | (5.43) | (10.77) |
| 19. | Co(D-leucinamide)(H₂O)₂Cl₂ | 24.74 | 5.62 | 9.25 |
| | | (24.34) | (5.45) | (9.47) |
| 20. | [Co(L- | 24.57 | 4.28 | 11.01 |
| | glutamine)Cl].½H₂O | (24.17) | (4.06) | (11.27) |
| 21. | [Co(D- | 23.87 | 4.22 | 10.75 |
| | glutamine)Cl].H₂O | (23.32) | (4.31) | (10.88) |
| 22. | [Co(L-glutamine-α- | 18.60 | 4.19 | 12.28 |
| | amide)Cl₂].3H₂O | (18.25) | (4.58) | (12.77) |
| 23. | Co(L-tyrosineamide)(CO₃)H₂O | 37.16 | 4.22 | 8.7.1 |
| | | (37.64) | (4.45) | (8.80) |
| 24. | Co(ethylenediamine)(OH)Cl | 14.54 | 5.50 | 16.29 |
| | | (14.00) | (5.29) | (16.33) |
| 25. | Co(1,2-phenylenediamine)Cl₂ | 30.28 | 3.69 | 11.81 |
| | | (30.27) | (3.39) | (11.77) |
| 26. | [Co(N- | 43.03 | 4.56 | 8.00 |
| | naphthylethylenediamine)Cl₂].H₂O | (43.13) | (4.79) | (8.38) |
| 27. | [Co[(Meso-1,2-diphenyl)ethylene- | 48.41 | 5.17 | 7.85 |
| | diamine]Cl₂].½H₂O | (47.84) | (4.84) | (7.97) |
| 28. | Co(2,2′-dipyridyl)Cl₂ | 41.77 | 2.75 | 9.62 |
| | | (41.98) | (2.82) | (9.79) |
| 29. | Co(1,10-phenanthroline)Cl₂ | 46.36 | 2.47 | 8.90 |
| | | (46.48) | (2.60) | (9.04) |
| 30. | Co(trans-1,2- | 28.49 | 5.73 | 11.10 |
| | DACH)Cl₂].½H₂O | (28.48) | (5.97) | (11.07) |
| 31. | [Co[(R,R-1,2-diphenyl)ethylene- | 51.95 | 5.12 | 8.70 |
| | diamine]Cl₂]; ½H₂O | (51.78) | (5.54) | (8.63) |
| 32. | Co(Salicylate)₂(H₂O)₂ | 45.73 | 4.21 | 0.00 |
| | | (45.54) | (3.82) | (0.00) |
| 33. | Co(Salicylhydroxamate)₂Cl₂ | 38.45 | 3.48 | 6.12 |
| | | (38.45) | (3.24) | (6.42) |
| 34. | Co(Thiosalicylate)₂(H₂O)₂ | 34.28 | 2.91 | 0.00 |
| | | (34.03) | (3.26) | (0.00) |
| 35. | Co[N,N-bis(salicylidene) | | | |
| | ethylenediamine] | | | |
| 36. | Co[N,N-bis(salicylidene)1,2- | | | |
| | phenylenediamine] | | | |
| 37. | Co(triphenylphosphine)₂Cl₂ | | | |
| 38. | Co[1,2-bis(diphenylphosphino)ethane]Cl₂ | | | |
| 39. | Co[N,N-bis(salicylidene)dianiline] | | | |
| 40. | Co[bis(salicylideniminaato-3- | | | |
| | propyl)methylamine] | | | |
| 41. | Co(acetylacetanote)₂ | | | |
| 42. | Co(hexafluoroacetylacetonate)₂ | | | |
| 43. | Co(benzoylacetonate)₂ | | | |
| 44. | Co(salicylaldehyde)₂ | | | |

Testing compounds for biochemical activity

Recombinant human Src was expressed using the baculovirus-insect cell system and purified as published (Budde et al., 1993). Recombinant Csk and the FGF receptor (FGFr) were expressed as glutathione-S-transferase fusion proteins using the pGEX expression vector and E. coli, and purified as described (Sun et al., 1995).

The tyrosine kinase activity of Src, Csk and FGFr was determined using poly E₄Y and ³²P-ATP. Briefly, enzymes were assayed in a reaction mixture consisting of 0.15 M EPPS-NaOH (pH 8.0) with 6 mM MgCl₂, 0.2 mM γ³²P-ATP (0.2-0.4 mCi/μmol), 10% glycerol, 0.1% Triton X-100, and poly E₄Y. Poly E₄Y is a synthetic peptide whose phosphorylation is measured in this assay by the addition of the radioactively labeled phosphate from the ATP (Budde et al., 1994). For screening assays, 50 μg/ml poly E₄Y was used.

Cobalt (II) complexes were identified as especially good inhibitors of Src or FGFr if they possessed an IC₅₀ of 10 μM or less, although higher values establish promising potential that warrants future investigation. One or more disclosed cobalt complexes in the category of especially good inhibitors of Src and/or FGFr include complex nos. 10, 11, 25, 27, 33 and 36. However, all of the disclosed complexes have excellent potential and numerous other commercial candidates will emerge after further experimentation.

TABLE 2
PTK Inhibition Data of Synthesized Cobalt(II) Complexes

| Complex No. | IC50(μM) | | |
|---|---|---|---|
| (See Table 1) | Src | Csk | FGFr |
| 1. | 26 | 53 | 14 |
| 2. | 46 | 196* | 183* |
| 3. | 144* | NI | NI |
| 4. | 176* | NI | NI |
| 5. | 259* | NI | NI |
| 6. | NI | NI | NI |
| 7. | NI | NI | NI |
| 8. | 100 | 150* | NI |
| 9. | NI | NI | NI |
| 10. | 2 | NI | 5 |
| 11. | 4 | 33 | 3 |
| 12. | NI | NI | NI |
| 13. | 24 | 77 | 54 |
| 14. | 28 | NI | 20 |
| 15. | 46 | 330* | 180* |
| 16. | 285* | NI | NI |
| 17. | 56 | 218* | 22 |
| 18. | 105* | 211* | 38 |
| 19. | 35 | NI | 45 |
| 20. | 57 | 281* | 100 |
| 21. | 58 | 389* | 93 |
| 22. | 71 | 361* | 46 |
| 23. | NI | NI | NI |
| 24. | 215* | NI | 24 |
| 25. | 9 | 210* | 14 |
| 26. | 65 | NI | 39 |
| 27. | 7 | 59 | 17 |
| 28. | NI | NI | NI |
| 29. | NI | NI | NI |
| 30. | 79 | NI | 123* |
| 31. | 55 | 50 | 98 |
| 32. | NI | NI | 182 |
| 33. | 6 | 92 | 2 |
| 34. | NI | NI | 125 |
| 35. | 108 | 92 | 3 |
| 36. | 28 | 128* | 1 |
| 37. | NI | NI | 76 |
| 38. | 66 | NI | NI |
| 39. | 133* | NI | 155* |
| 40. | 95 | NI | 41 |
| 41. | 177* | NI | 233* |
| 42. | 105 | NI | 99 |
| 43. | 47 | NI | 83 |
| 44. | 113 | NI | 148* |

Note:
NI = No inhibition at 50 ug/ml; and
*= Extrapolated value from the graph.

Structures
a. General Structure of Formula 1 Cobalt(II) Complexes of Amino acid Hydroxamates and Amides

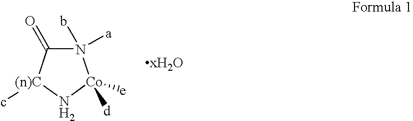

The following Table 3 provides examples of Formula 1 complexes. It will be noted that this disclosure as it pertains to Formula 1 complexes is not limited to the examples listed in Table 3 below.

TABLE 3
Examples of Formula 1 Complexes

| Complex | | | | | | | |
|---|---|---|---|---|---|---|---|
| No | a | b | c | d | e | n | X |
| 1 | H | OH | 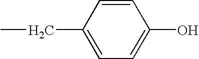 | ClO₄ | ClO₄ | 1 | 2 |
| 2 | H | H | 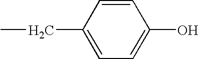 | ClO₄ | OH | 1 | 2 |
| 3 | H | H | 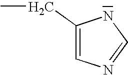 | Imidazole ‘N’ | ClO₄ | 1 | 1 |
| 4 | — | OH | —CH₂.CH₂—S—CH₃ | Methionyl | OH | 1 | 1 |
| | | | | ‘S’ | | | |
| 5 | H | OH | H | OH | OH | 1 | 0 |
| 6 & 7 | — | OH | 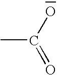 | Carboxylic ‘O’ | H₂O | 2 | 2 |
| 8 | H | OH | H | OH | OH | 2 | 1 |
| 9 | H | OH | —CH₃ | OH | OH | 1 | 0 |
| 10 & 11 | H | OH | 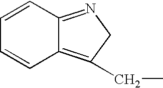 | Tryptophenyl ‘N’ | ClO₄ | 1 | 1 |
| 12 | — | OH | 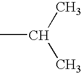 | Threonyl ‘O’ | H₂O | 1 | 1 |
| 13 | H | H | 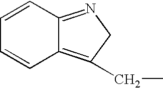 | ClO₄ | ClO₄ | 1 | 2 |
| 14 | H | H | 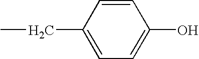 | Cl | Cl | 1 | 3 |
| 15 | H | H | 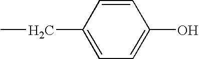 | OH | Cl | 1 | 1.5 |
| 16 | H | H | 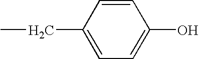 | OH | 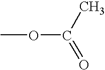 | 1 | 0 |
| 17 | H | H | H | Cl | Cl | 1 | 2 |
| 18 | H | H | 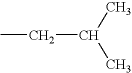 | Cl | Cl | 1 | 0 |
| 19 | H | H | 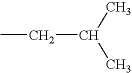 | Cl | Cl | 1 | 2 |
| 20 & 21 | H | H | 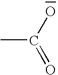 | Carboxylic ‘O’ | Cl | 3 | ½ |
| 22 | H | H | 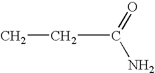 | Amide ‘NH₂’ | Cl2 | 1 | 3 |
| 23 | H | H | 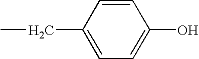 | Carbonate ‘O’ | Carbonate ‘O’ | 1 | 1 |

b. General Structure of Formula 2 Cobalt(II) Complexes of Ethylenediamine and Other Derivatives

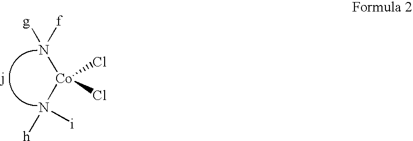

The following complex nos. 24-31 are examples of Formula 2 complexes. It will be noted that this disclosure as it pertains to Formula 2 complexes is not limited complex nos. 24-31.

Complex No. 24: f═g═h═i═H, and j is —CH₂CH₂—;

Complex No. 25: f═g═h═i═H, and

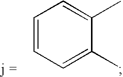

Complex No .26: f═g═h═H, 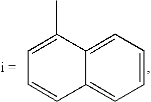
and j is —CH₂CH₂—;

Complex Nos. 27 and 31: f═g═h═i═H, and

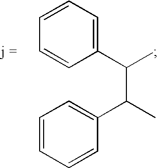

Complex No.28: f═g═h═i═—, and

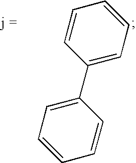

Complex No.29: f═g═h═i═—, and

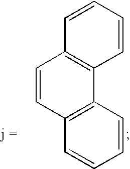

Complex No. 30: f═g═h═i═H, and 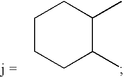
C. General Structure of Formula 3 Cobalt(II) Complexes of Salicylic Acid Derivatives

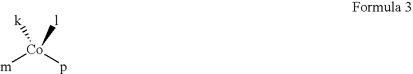

The following complexe nos. 32 and 33 are examples of Formula 3 complexes. It will be noted that this disclosure as it pertains to Formula 3 complexes is not limited to complex nos. 32 and 33.

Complex No. 32: k═l═H₂O, and

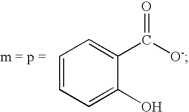

Complex No. 33: k═l═Cl, and 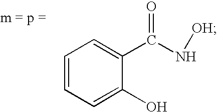
d. General Structure of Formula 4 Cobalt(II) Complexes Purchased from Aldrich Chemical Co.

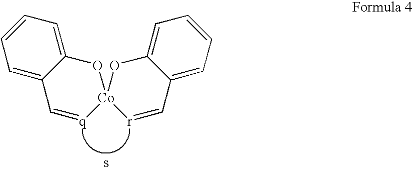

The following are mere examples of Formula 4 complexes and this disclosure, as it pertains to Formula 4 complexes is not limited to the specific examples listed below.

Compound No. 35: q═r═N, and s is —CH₂CH₂—;

Compound No. 36: q═r═N, and

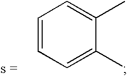

Compound No. 39:

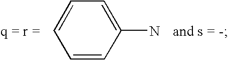

Compound 44, q═r═O, and s ═ —.

### References

1. Barnekow, A. Crit. Rev. Oncogenesis 1989, 1, 277-292.
2. Bjelfman, C., Hedborg, R., Johnsson, I., Nordenskjold, M., and Pahlman, S. Cancer Res. 1990, 50, 6908-6914.
3. (a) Bowie, E. A., and Hurley, P. J., Aust. N. Z. J. Med. 1975, 5, 306-314; (b) Valberg, L. S. Lancet 1971, 2, 1092; and (c) Edwards, M. S., and Curtis, J. R. Lancet 1971, 2, 582-583.
4. Budde, R., Ke, S., and Levin, V., Cancer Biochem. Biophys. 1994, 14, 171-175.
5. Budde, R., Ramdas L, and Ke S: “Recombinant Src from Baculovirus-infected Insect Cells: Purification and Characterization,” Preparative Biochemistry 23:493-515, 1993.
6. Budde, R., Ke, S, and Levin, V., “Activity of pp60c-src in 60 Different Cell Lines Derived from Human Tumors,” Cancer Biochem. Biophys. 14:171-175, 1994.
7. Cartwright, C. A., Meisler, A. I., and Eckhart, W. Proc. Natl. Acad. Sci. USA. 1990, 87, 558-562.
8. Cooley, J., Kamaras, P., and Rapta, M. Acta Cryst. 1995, C51, 1811-1813.
9. Curtis, J., Goode, G., Herrington, J., and Urdaneta, L., Clin. Nephrol. 1976, 5, 61-65.
10. Dancey, J., and Sausville, E., Nature Drug Disc. Rev. 2003, 2, 296-299.
11. Drugs of the Future 1996, 21, 1215-1220.
12. Fanning, P., Bulovas, K., Saini, K., Libertino, J., Joice, A., and Summerhayes, I., Cancer Res. 1992, 52, 1457-1462.
13. Frame, M., Biochim. et Biophys. Acta 2002, 1602, 114-130.
14. (a) Hall, M., Failes, T., Hibbs, D., and Hambley, T., Inorg. Chem. 2002, 41, 1223-1228; (b) Brown, D., Errington, W., Glass, W., Haase, W., Kemp, T., Nimir, H., Ostrovsky, S., and Warner, R. Inorg. Chem. 2001, 40, 5962-5971; and (c) O'Brien, E., Franks, E., Gil, M., Fitzgerald, D., Castineras, A., and Nolens, K. D. J. Inorg. Biochem., 2000, 79, 47-51.
15. Irby, R., and Yeatman T., Oncogene, 2000, 19, 5636-5642.
16. Jessup, J., and Gallick, G., Curr. Problems in Cancer 1993, 16, 263-328.
17. Judd, D., Nettles, J., Nevins, N., Snyder, J., Liotta, D., Tang, J., Ermolieff, J., Schinazi, R., and Hill, C., J. Am. Chem. Soc. 2001, 123, 886-897.
18. Kitanaka, A., Waki, M., Kamono, H., and Tanaka, T. Biochem. Biophys. Res. Commun. 1994, 201, 1534-1540.
19. Lynch, S., Brugge, J., Fromowitz, F., Glantz, L., Wang, P., Caruso, R., and Viola, M., Leukemia 1993, 7, 1416-1422.
20. Park, S., Kang, S., Lim, S., Oh, S., and Lee, K., Bioorg. Med. Chem. Lett. 2003, 13, 3455-3459.
21. Partanen, S. Oncol. Repts. 1994, 1, 603-606.
22. Sariano, P., Montogomery, C., Geske, R., and Bradley, A., Cell, 1991, 64, 693-702.
23. Sinha, S., and Corey, S., J. Hematotherapy & Stem Cell Res. 1999, 8, 465-480.
24. Sridhar, R., Hanson-Painton, O., and Cooper, D., Pharm Res. 2000, 17, 1345-1353.
25. Staley, C., Parikh, N., and Gallick, G., Cell Growth & Differen. 1997, 8, 269-274.
26. Sun, G., and Budde, R., Biochemistry 1997, 36, 2139-2146.
27. Sun, G., and Budde, R., Biochemistry 1999, 38, 5659-5665.
28. Sun G and Budde R., “A modified pGEX Expression System that Eliminates Degradation Products and Thrombin from the Recombinant Protein,” Analytical Biochem., 231:458-460, 1995.
29. Susa, M., Missbach, M., and Green, J., Trends in Pharm. Sci., 2000, 21, 489-495.
30. Takeshima, E., Hamaguchi, M., Watanabe, T., Akiyama, S., Kataoka, M., Ohnishi, Y., Xiao, H., Nagai, Y., and Takaagi, H., Japan J. Cancer Res., 1991, 82, 1428-1435.
31. Termuhlen, P., Curley, S., Talamonti, M., Saboorian, M., and Gallick, G., J. Surg. Res. 1993, 54, 293-298.
32. Waki, M., Kitanaka, A., Kamano, H., Tanaka, T., Kubota, Y., Ohnishi, H., Takahara, J., and Irino, S. Biochem. Biophys. Res. Commun. 1994, 201, 1001-1007.
33. Wiener, J., Nakano, K., Kruzelock, R., Bucana, C., Bast, R., and Gallick, G., Clin. Cancer Res. 1999, 5, 2164-2170.

What is claimed:

1. A complex selected from the group consisting of the following Formulas 1 through 4: 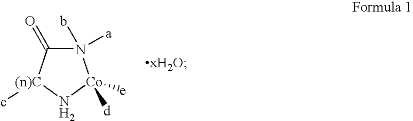
wherein a is H or —;
wherein b is H or OH;
wherein c is selected from the group consisting of H, —CH₃, 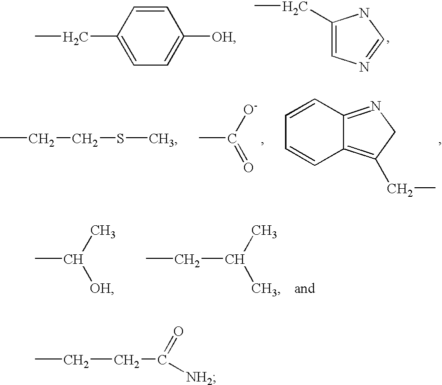
wherein d is selected from the group consisting of OH, ClO₄, imidazole, methionyl, carboxylic, tryptophenyl, threonyl, amide and carbonate;
wherein e is selected from the group consisting of OH, ClO₄, H₂O, Cl, 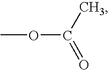
Cl₂, and carbonate;
wherein n is an integer ranging from 1 to 3; and
wherein x ranges from 0 to about 3; 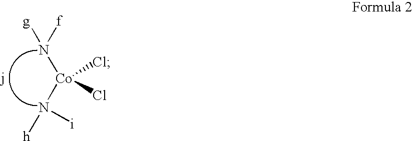
wherein f, g, and h are H or —;
wherein i is selected from the group consisting of H, —, and 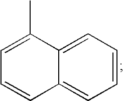
and
wherein j is selected from the group consisting of —CH₂CH₂—, 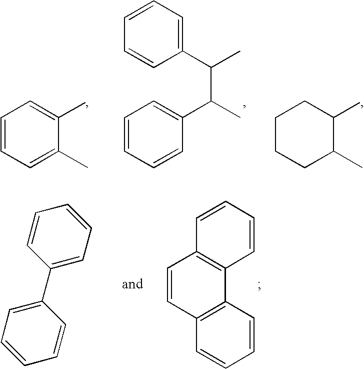 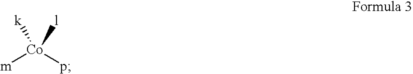
wherein k and l are H₂O or Cl; and
wherein m and p are selected from the group consisting of 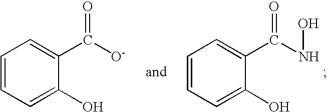 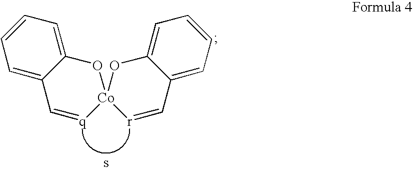
wherein q and r are selected from the group consisting of N, O and 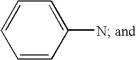
wherein s is selected from the group consisting of —CH₂CH₂—, —, and

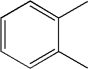

2. The complex of claim 1 wherein the complex is selected from the group consisting of: Co(L-Tyrosinehydroxamate)(H₂O)₂(ClO₄)₂;
Co(L-Tyrosine amide)(H₂O)₄(ClO₄); Co(L-Histidinehydroxamate)(H₂O)(ClO₄);
Co(DL-Methioninehydroxamate)(OH)H₂O;
Co(Glycinehydroxamate)(OH)₂; Co(DL-Asperticacid-β-hydroxamate)(H₂O)₃;
Co(L-Asperticacid-β-hydroxamate)(H₂O)₃; Co(β-alaninehydroxamate)(OH)₂; Co(DL-alaniniehydroxamate)(OH)₂;
Co(L-tryptophanhydroxamate)(H₂O)(ClO₄); Co(D-tryptophanhydroxamate)(H₂O)(ClO₄);
Co(L-Thrioooninehydroxamate)(OH)(H₂O); Co(L-Tryptophanamide)(H₂O)₂(ClO₄)₂;
Co(L-Tyrosineamide)(H₂O)₃Cl₂;
[Co(L-Tyrosine amide)(OH)Cl].1½H₂O; Co(L-Tyrosine amide)(Acetate)(OH);
[Co(glycinamide)(H₂O)₂Cl₂].NaCl; Co(L-Leucinamide)Cl₂;
CO(D-leucinamide)(H₂O)₂Cl₂; [Co(L-glutamine)Cl].1/2 H₂O;
[Co(D-glutamine)Cl].H₂O; [Co(L-glutamine-α-amide)Cl₂].3H₂O;
Co(L-tyrosineamide)(CO₃)H₂O; Co[N,N-bis(salicylidene)ethylenediamine];
Co[N,N-bis(salicylidene)1,2-phenylenediamine];
Co(triphenylphosphine)₂Cl₂;
Co[1,2-bis(diphenylphosphino)ethane]Cl₂;
Co[N,N-bis(salicylidene)dianiline];
Co[bis(salicylideniminaato-3-propyl)methylamine];
Co(acetylacetanote)₂;
Co(hexafluoroacetylacetonate)₂;
Co(benzoylacetonate)₂;
Co(salicylaldehyde)₂;
Co(ethylenediamine)(OH)Cl;
Co(1,2-phylenediamine)Cl₂;
[Co(N-naphthylethylenediamine)Cl₂].H₂O;
{Co[(Meso-1,2-diphenyl)ethylene-diamine]Cl₂}.½H₂O;
Co(2,2′-dipyridyl)Cl₂; Co(1,10-phenanthroline)Cl₂;
[Co(trans-1,2-DACH)Cl₂].½H₂O;
{Co[(R,R-1,2-diphenyl)ethylene-diamine]Cl₂}.½H₂O;
Co(Salicylate)₂(H₂O)₂; Co(Salicylhydroxamate)₂Cl₂;
Co(Thiosalicylate)₂(H₂O)₂; and mixtures thereof.

3. The complex of claim 1 wherein the complex has the structure of Formula 1.

4. The complex of claim 3 wherein a is H, b is OH, 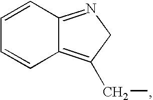
d is Tryptophenyl‘N’ and e is ClO₄.

5. The complex of claim 1 wherein the complex has the structure of Formula 2.

6. The complex of claim 5 wherein f, g, h and i are all H, and

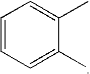

7. The complex of claim 5 wherein f, g, h and i are all H, and

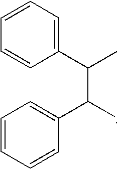

8. The complex of claim 1 wherein the complex has the structure of Formula 3.

9. The complex of claim 8 wherein k and l are Cl,

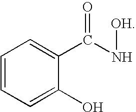

10. The complex of claim 1 wherein the complex has the structure of Formula 4.

11. The complex of claim 10 wherein q and r are N, and

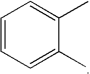

12. A pharmaceutical composition comprising a carrier and at least one complex of claim 1.

13. The pharmaceutical composition of claim 12 wherein the complex is selected from the group consisting of:
Co(L-Tyrosinehydroxainate)(H₂O)₂(ClO₄)₂;
Co(L-Tyrosine amide)(H₂O)₄(ClO₄); Co(L-Histidinehydroxamate)(H₂O)(ClO₄);
Co(DL-Methioninehydroxamate)(OH)H₂O;
Co(Glycinehydroxamate)(OH)₂; Co(DL-Asperticacid-β-hydroxamate)(H₂O)₃;
Co(L-Asperticacid-β-hydroxamate)(H₂O)₃; Co(β-alaninehydroxamate)(OH)₂; Co(DL-alaniniehydroxamate)(OH)₂;
Co(L-tryptophanhydroxamate)(H₂O)(ClO₄); Co(D-tryptophanhydroxamate)(H₂O)(ClO₄);
Co(L-Thrioooninehydroxamate)(OH)(H₂O); Co(L-Tryptophanamide)(H₂O)₂(ClO₄)₂;
Co(L-Tyrosineamide)(H₂O)₃Cl₂;
[Co(L-Tyrosine amide)(OH)Cl].1½H₂O; Co(L-Tyrosine amide)(Acetate)(OH);
[Co(glycinamide)(H₂O)₂Cl₂].NaCl; Co(L-Leucinamide)Cl₂;
Co(D-leucinamide)(H₂O)₂Cl₂; [Co(L-glutamine)Cl].1/2 H₂O;
[Co(D-glutamine)Cl].H₂O; [Co(L-glutamine-α-amide)Cl₂].3H₂O;
Co(L-tyrosineamide)(CO3)H₂O; Co[N,N-bsi(salicylidene)ethylenediamine];
Co[N,N-bis(salicylidene) 1,2-phenylenediamine];
Co(triphenylphosphine)₂Cl₂;
Co[1,2-bis(diphenylphosphino)ethane]Cl₂;
Co [N,N-bis(salicylidene)dianiline];
Co[bis(salicylideniminaato-3-propyl)methylamine];
Co(acetylacetanote)₂;
Co(hexafluoroacetylacetonate)₂;
Co(benzoylacetonate)₂;
Co(salicylaldehyde)₂;
Co(ethylenediamine)(OH)Cl;
Co(1,2-phylenediamine)Cl₂;
[Co(N-naphthylethylenediamine)Cl₂].H₂O;
{Co[(Meso-1,2-diphenyl)ethylene-diamine]Cl₂}.½H₂O;
Co(2,2′-dipyridyl)Cl₂; Co(1,10-phenanthroline)Cl₂;
[Co(trans-1,2-DACH)Cl₂].½H₂O;
{Co[(R,R-1,2-diphenyl)ethylene-diamine]Cl₂}.½H₂O;
Co(Salicylate)₂(H₂O)₂; Co(Salicylhydroxamate)₂Cl₂;
Co(Thiosalicylate)₂(H₂O)₂; and mixtures thereof.

14. A method of inhibiting a protein tyrosine kinase by administering a subject at least one complex defined in claim 1.

15. The method of claim 14 comprising the step of the binding of the complex to said protein tyrosine kinase.

16. The method of claim 14 wherein the protein tyrosine kinase is selected from the group consisting of Src, Fynn, Yes, Lyn, Lck, Blk, Hck, and Fgr.

17. The method of claim 14 wherein the subject is a mammal.

18. The method of claim 17 wherein the mammal is a human.

19. The method of claim 23 wherein the administering is parenteral, alimentary, topical or by inhalation.
